(19) United States
(12) Patent Application Publication
(10) Pub. No.: US 2026/0192156 A1
(43) Pub. Date: Jul. 9, 2026

(54) WEARABLE DEVICE SYSTEMS AND METHODS FOR PROGRESSIVE LOADING

(71) Applicant: Xperience Robotics, Inc., Virginia Beach, VA (US)

(72) Inventor: Ankit SHAH, Virginia Beach, VA (US)

(21) Appl. No.: 19/440,098

(22) Filed: Jan. 5, 2026

Related U.S. Application Data
(60) Provisional application No. 63/742,075, filed on Jan. 6, 2025.

(51) Int. Cl. A63B 24/00

(52) U.S. Cl. CPC A63B 24/0062; A63B 24/0003; A63B 2220/20; A63B 2220/34; A63B 2220/51

(57) ABSTRACT

A system for progressive load monitoring with wearable devices is provided. The system includes a memory storing instructions and one or more processors that, when executing the instructions, perform operations including: receiving first information indicative of a type of motion of a user to be monitored with one or more wearable devices, where the type of motion includes motion performed by interaction with a motion-resisting device that resists the motion; receiving, via one or more sensors of the one or more wearable devices, second information indicative of a speed and a direction of the motion; analyzing the motion based on the first and second information; and determining, based on the analysis, whether the motion satisfies a predetermined motion target including at least one of a target range of speeds or a threshold number of repetitions associated with an amount of resistance generated by the motion-resisting device.

### CROSS-REFERENCE TO RELATED APPLICATION(S

This application claims the benefit of priority to U.S. Provisional Application No. 63/742,075, filed on January 6, 2025, the entirety of which is incorporated by reference.

### BACKGROUND

Some wearable devices are equipped with sensors that provide information related to an individual’s physical motion. These devices can monitor, track, and log movements, as well as track certain physiological data such as step count or heart rate. However, these devices and the systems that use them are limited in that they cannot assess the performance of a movement or exercise while taking into account factors, such as load or tension on the subject.

### SUMMARY

According to aspects of the disclosure, methods and systems are disclosed for progressive load monitoring of a motion of a user. For example, a system for progressive load monitoring with wearable devices may include a memory storing instructions and one or more processors that, when executing the instructions, perform operations including: receiving first information indicative of a type of motion of a user to be monitored with one or more wearable devices, where the type of motion includes motion performed by interaction with a motion-resisting device that resists the motion; receiving, via one or more sensors of the one or more wearable devices, second information indicative of a speed of the motion and a direction of the motion; analyzing the motion based on the first information and the second information; and determining, based on the analysis, whether the motion satisfies a predetermined motion target, where the predetermined motion target includes at least one of a target range of speeds or a threshold number of repetitions associated with an amount of resistance generated by the motion-resisting device.

In some examples, a method for progressive load monitoring with wearable devices may include receiving first information indicative of a type of motion of a user to be monitored with one or more wearable devices, where the type of motion includes motion performed by interaction with a motion-resisting device that resists the motion; receiving, via one or more sensors of the one or more wearable devices, second information indicative of a speed of the motion and a direction of the motion; analyzing the motion based on the first information and the second information; and determining, based on the analysis, whether the motion satisfies a predetermined motion target, where the predetermined motion target includes at least one of a target range of speeds or a threshold number of repetitions associated with an amount of resistance generated by the motion-resisting device.

In some examples, a non-transitory computer readable storage medium, having instructions stored therein, which when executed by one or more processors, cause the one or more processors to: receive first information indicative of a type of motion of a user to be monitored with one or more wearable devices, where the type of motion includes motion performed by interaction with a motion-resisting device that resists the motion; receive, via one or more sensors of the one or more wearable devices, second information indicative of a speed of the motion and a direction of the motion; analyze the motion based on the first information and the second information; and determine, based on the analysis, whether the motion satisfies a predetermined motion target, where the predetermined motion target includes at least one of a target range of speeds or a threshold number of repetitions associated with an amount of resistance generated by the motion-resisting device.

Additional objects and advantages of the disclosed embodiments will be set forth in part in the description that follows, and in part will be apparent from the description, or may be learned by practice of the disclosed embodiments.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 illustrates a diagram of an environment where a progressive load monitoring system can be used with one or more wearable devices and an exercise device, according to an example of the present disclosure.

FIG. 2 illustrates a user with wearable devices using an exercise device, according to an example of the present disclosure.

FIGS. 3A and 3B illustrate a user with wearable devices using another exercise device, according to an example of the present disclosure.

FIG. 4 is a flowchart of an exemplary method for progressive load monitoring of a motion of a user.

### DETAILED DESCRIPTION

Reference will now be made in detail to the present examples, including examples illustrated in the accompanying drawings. Wherever possible, the same reference numbers will be used throughout the drawings to refer to the same or like parts.

Reference numerals and terminology used herein are for the purpose of describing particular aspects only and are not intended to be limiting. For example, as used herein, the singular forms “a,” “an” and “the” are intended to include the plural forms as well, unless the context clearly indicates otherwise. Also, the term “or” is intended to encompass “and/or” when used in reference to two or more alternatives, unless stated otherwise. As another example, recitation of ranges of values herein are merely intended to serve as a shorthand method of referring individually to each separate value falling within the range. Unless otherwise indicated herein, and each separate value is incorporated into the specification as if it were individually recited herein.

It is noted that any one or more aspects or features described with respect to one example, may be incorporated in different examples described herein, although not specifically referred to or otherwise described relative thereto. That is, all examples and/or features of any aspect described herein can be combined in any way and/or combination. Thus, all methods described herein can be performed in any suitable order unless otherwise indicated herein or otherwise clearly contradicted by context.

Any examples discussed in the present disclosure can be incorporated into a non-transitory, computer-readable medium having instructions that, when executed by a processor associated with a computing device, cause the processor to perform the stages described. Additionally, any example methods discussed in the present disclosure can be implemented in a system including, for example, a memory storage and a computing device having a processor that executes instructions to carry out the steps described herein.

Aspects of the present disclosure provide systems and methods for progressive load monitoring of a motion of a user. For example, using the system according to an example of the present disclosure, a user with a wearable device or sensor may perform prescribed movements. The system may monitor the motion of the user via one or more sensors, and provide a feedback to the user in real-time. The user performance data may be transmitted to the system (and ultimately to the doctor, clinician, or user), and based on the user performance data, the system may generate a new prescribed movement.

Moreover, based on the user performance data, the system may display an identification of, or otherwise recommend, an exercise device (e.g., resistance bands or weights) for the user. For example, based on the user performance data, the system may recommend a type of exercise device (e.g., adjusting the level of resistance or weight, suggesting a different exercise device) that would be most suitable for the user. In some examples, information about the exercise device (e.g., resistance bands or weights) to be used by the user may be provided as an input to the system.

In some examples, as the user performs the (prescribed) movement (e.g., according to the displayed identification, recommendation, or guidance from the system) with the exercise device (e.g., resistance bands or weights), the system may monitor the user’s movement/motion (via wearable devices or sensors), and check the correctness of the movement/motion (e.g., form, movement trajectory, speed, number of repetitions).

In some examples, based on the monitored or checked data, the system may be able to determine or calculate the external force being applied by the exercise device. For example, the system, based on its algorithm, may associate the relationship between the exercise device and the user’s movement or motion being performed, and provide a real-time feedback to the user, for example, to improve the user’s form or movement and/or increase/decrease the speed of the movement or motion.

In some examples, the system may correlate the force or tension of the movement being performed (assuming that the force value is generated by the exercise device) with a baseline of the same movement without the influence or use of the exercise device. In some examples, the system may determine/calculate the applied force by the user for all or partial body parts (e.g., joints, limbs, or muscles), while the respective body parts are under the influence of external force applied by the movement using the exercise device. This force value can be used as an indicator of the user’s strength level.

In some examples, based on the monitored and calculated data related to the user’s movements or motions (e.g., the movement metrics, such as sets and repetitions of the movements, a range of motions, speed, acceleration, rotational velocity, etc.), the system may determine a strength level of the user. For example, the system may analyze the user’s movements or motions in three dimensions (e.g., along the x, y, z axes), and calculate or otherwise determine related parameters (e.g., acceleration, rotational velocity, etc.) in each axis or plane. In some examples, the system may measure all of the user movement metrics for each strength level, and determine or otherwise calculate a pain score for each strength level. The system may also transmit the monitored and calculated data to the clinician, doctor, or user.

In some examples, based on the monitored and calculated data related to the user’s movements or motions, the system may determine whether to advance or lower the strength level (force, resistance, or weight) of the user. In some examples, the system may perform the monitoring and feedback or recommendation generation steps in real-time.

As used herein, the term “real-time,” “real time,” and “substantially real-time” are used to refer to processing performed within time constraints. For example, as described herein, real-time tracking, collection, processing, and delivery of raw sensor data, formatted data, comparative analysis data, and feedback: without a perceivable delay or within 5 seconds of the event associated with the data.

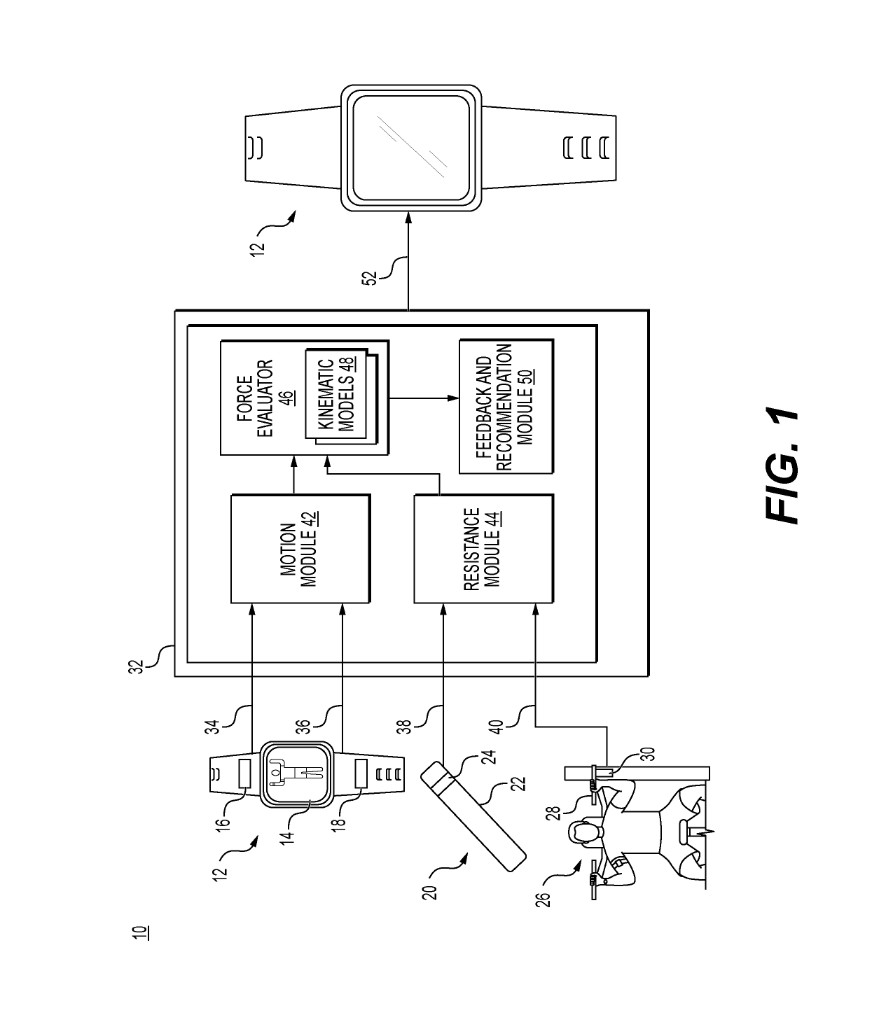

FIG. 1 illustrates a diagram of an environment 10 where a progressive load monitoring system 32 can be used, according to an example of the present disclosure. In some embodiments, the progressive load monitoring system 32 may include a motion module 42, a resistance module 44, a force evaluator 46, and a feedback and recommendation module 50.

The motion module 42 of the progressive load monitoring system 32 may be in communication with one or more wearable devices 12. The wearable device 12 may include a display 14 and one or more sensors 16, 18. In some examples, the one or more sensors 16, 18 may include an acceleration sensor and a gyroscopic sensor. In other examples, the one or more sensors may include any other suitable sensors (e.g., temperature sensor, magnetometer, a hall sensor, optical encoders, and/or any form of optical sensor).

According to an aspect of the present disclosure, “wearable device” includes any form factor designed or intended to be worn by a person (e.g., personal equipment such as helmets, face guards, apparel, or the like; personal devices such as head-mounted electronic devices, wrist mounted devices, body-mounted devices, devices worn around the neck; or any form factor that, while not necessarily designed or intended to be worn by a person, may be adapted to be worn by a person (e.g., smartphones, tablet computers, and/or other digital processing devices).

Each wearable device may constitute a computing device including a processor, a memory storage, and a non-transitory computer-readable medium containing instructions that are executed by the processor. The one or more sensors of the wearable device may be in communication with the processor.

In some examples, the wearable device may include a component that authenticates biometric-information (“bio-metric component”), a pulse oximeter, a blood oxygen sensor, and/or an iris scanner. This biometric component or sub-component may acquire biometric information from a user and use the acquired biometric information to authenticate and permit the user to use the wearable device. The biometric component may acquire biometric information through a surface or portion of the wearable device that is particularly close to or in contact with the user. The biometric information may be defined as information associated with the user’s living body (e.g., pulse, fingerprint, movement profile of one portion of the user’s body, body temperature, etc.) the face, and the walking pattern. This configuration may biometrically identify and authenticate the user without requesting an action by the user.

In some examples, the display 14 of the wearable device 12 may be provided as a device’s interface with, and a vehicle for delivering feedback to, a user wearing the device. Examples of the display 14 may include an LCD or OLED screen and/or a touch sensitive screen. In some examples, the wearable device 12 may include LEDs, vibration motors, and/or a means of generating audio feedback. While wearable device 12 is shown as a watch having a face portion formed by display 14, as understood, any of the functions described with respect to wearable device 12 (e.g., functions described with respect to display 14, sensor 16, sensor 18, etc.) may be performed with another type of device, such as a different type of wearable device or a mobile device. Suitable mobile devices (e.g., mobile phones, tablets, etc.) may be used to display feedback, display instructions to a user (e.g., instructions to perform a particular exercise, instructions use resistance band 20 or resistance equipment 26 at a particular resistance or weight, etc.), or display any other data or information described herein.

In some examples, the wearable device 12 according to the present disclosure can transfer data to or receive data from another device (e.g., progressive load monitoring system 32, another wearable device, etc.), as well as perform the methods described herein for progressive load monitoring. In some examples, certain processing functions may be distributed over multiple devices. The assignment of functions to one device over another may depend on various factors, such as the device’s position, the designated movements in the movement plan, and other considerations.

The one or more wearable devices 12 according to the present disclosure can communicate with another device (e.g., another wearable device 12 or the progressive load monitoring system 32) using any wireless protocol (e.g., Bluetooth, Bluetooth Low Energy, Zigbee, Wifi, near-field communication, etc.).Wireless or wired communication may facilitate communication with, for example, a cloud-based storage or analytic system.

In some examples, the wearable device 12 may be configured to transmit one or more signals 34, 36 to the progressive load monitoring system 32. The one or more signals 34, 36 from the wearable device 12 may include data that is sensed, detected, or measured by the one or more sensors 16, 18. Examples of the data may include acceleration (e.g., changes in velocity over time) and angular velocity of a motion of a user wearing the wearable device 12, a body temperature of the user, and/or any other suitable data (e.g., data from one or more sensors of the wearable device 12).

In some examples, the resistance module 44 of the progressive load monitoring system 32 may be in communication with one or more exercise devices 20, 26. In some examples, the exercise devices 20, 26 may include a resistance band 20 and/or resistance equipment 26 (e.g., weight training device). In other examples, resistance module 44 is in communication with an input device (e.g., a touchscreen, keyboard, mouse, etc.) connected to progressive load monitoring system 32, wearable device 12, or to another system.

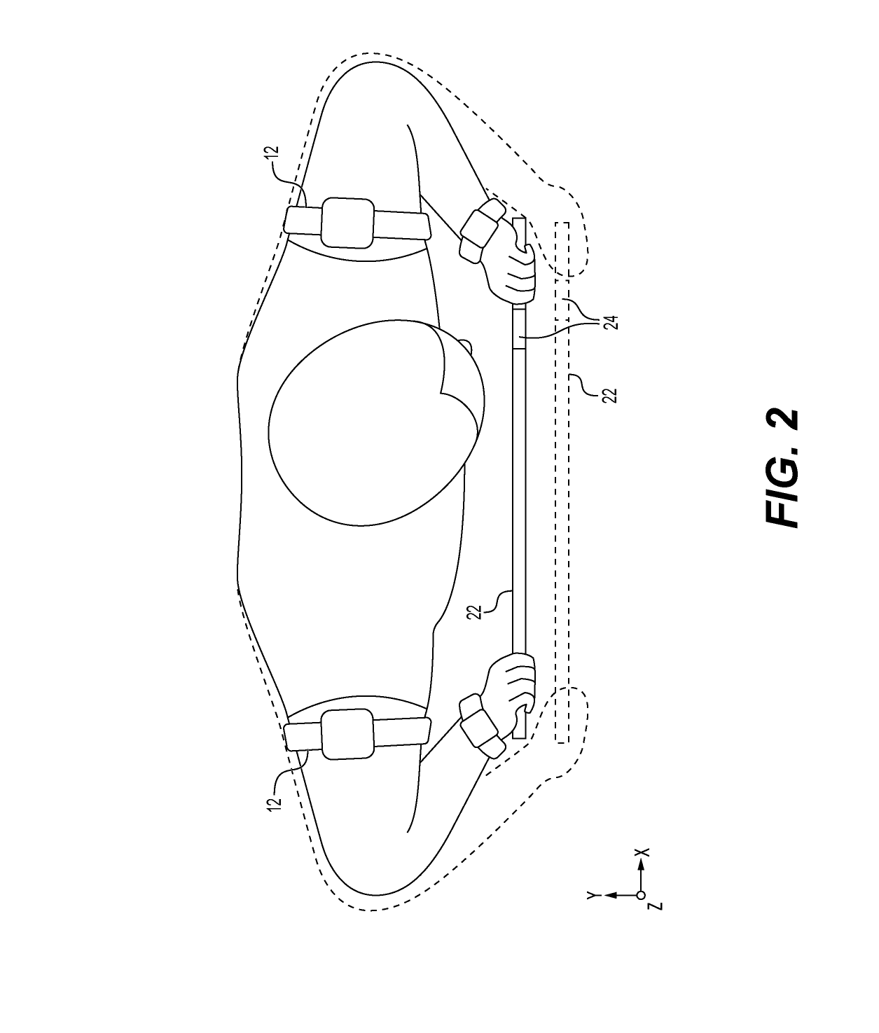

The resistance band 20 may include a resistive portion 22. The resistive portion 22 may be made of elastic materials (e.g., rubber, latex, fabric) and may be stretchable as shown in FIG. 2. The resistive portion 22 may provide resistance when stretched. For example, as the band stretches, the tension/resistance of the resistive portion 22 opposes motion of the user.

In some examples, the resistance band 20 may also include a resistance transmitter 24 and one or more sensors. The resistance transmitter 24 and one or more sensors may be embedded within or connected to the resistance band 20. The one or more sensors may monitor a magnitude of the force, tension, or resistance of the resistance band 20 and/or the number of times that the resistive portion 22 has been stretched (e.g., the number of times that the resistive portion 22 was stretched beyond a threshold distance). The resistance transmitter 24 of the resistance band 20 may be configured to transmit one or more signals 38 to the progressive load monitoring system 32 (e.g., resistance module 44). The one or more signals 38 from the resistance band 20 may include data sensed, detected, or measured by the one or more sensors of the resistance band 20. Examples of the data may include the magnitude of the force, tension, or resistance of the resistance band 20 and/or the number of times that the resistive portion 22 was stretched (e.g., the number of times that the resistive portion 22 was stretched over a threshold distance). In some examples, an RFID tag or other resistance transmitter 24 is configured to output a signal that indicates the quantity of resistance that resistance band 20 is capable of. In other examples, the resistance of the resistance band 20 may be provided by the user as an input into the progressive load monitoring system 34.

In some examples, the user may wear the one or more wearable devices 12 on the user’s arm(s). For example, as shown in FIG. 2, the user may wear a first wearable device 12 on the left arm and a second wearable device 12 on the right arm, at different locations on the same limb, etc. The first and second wearable devices 12 may detect the speed, direction of the motion of the user while the user exercises using the resistance band 20 (e.g., stretching the resistance band 20 repetitively).

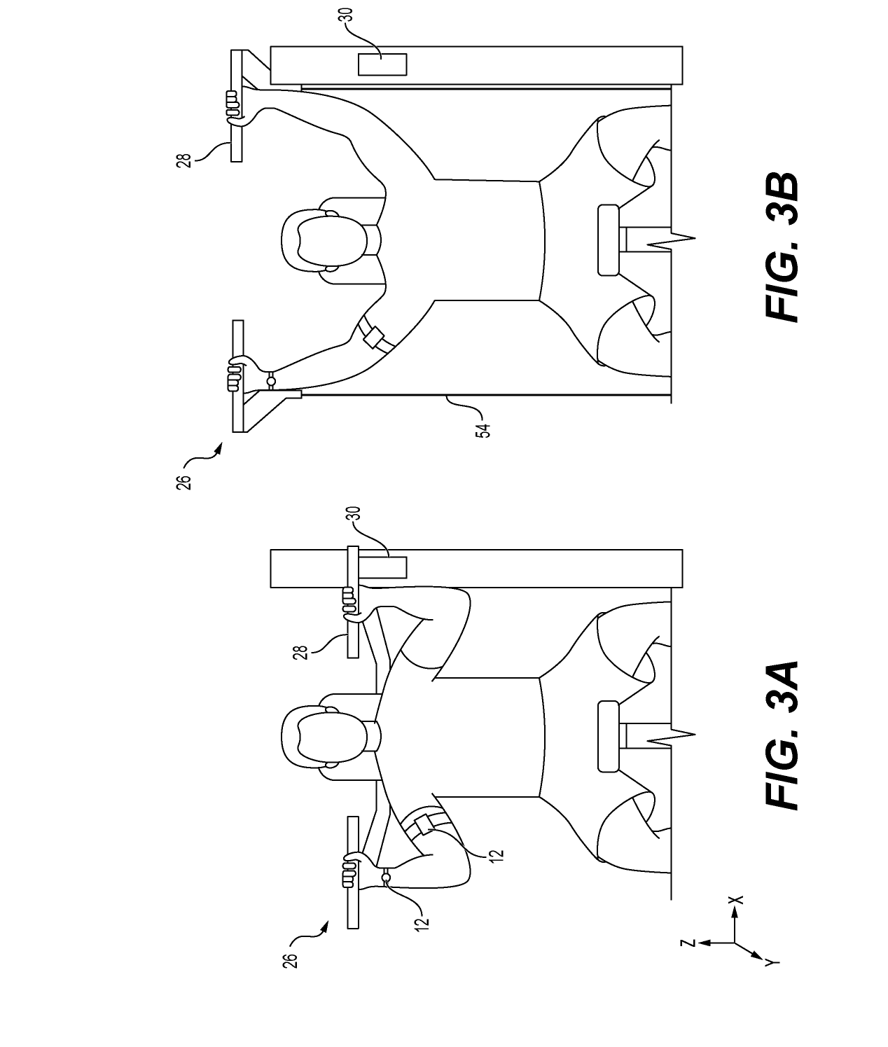

The resistance equipment 26 may include a moving portion 28. The moving portion 28 may include a weight and/or provide resistance or weight when a user tries to push, pull, lift, lower, or rotate the moving portion 28. For example, as shown in FIG. 3, a user may lift/push the moving portion 28 and lower or pull the moving portion 28 repetitively.

In some examples, the user may wear the one or more wearable devices 12 on the user’s arm or wrist. For example, as shown in FIGS. 3A and 3B, a user may wear a first wearable device 12 on the arm and a second wearable device 12 on the wrist. The first and second wearable devices 12 may detect the speed or direction of the motion of the user while the user exercises using the resistance equipment 26 (e.g., pushing, pulling, lifting, lowering, or rotating the moving portion 28 repetitively).

In some examples, the resistance equipment 26 may also include a resistance transmitter 30 and one or more sensors. The one or more sensors may monitor a magnitude of the force, tension, resistance, or weight of the resistance equipment 26 and/or the number of repetitions (e.g., the number of the moving portion 28 being pushed, pulled, lifted, lowered, or rotated). The resistance transmitter 30 of the resistance equipment 26 may be configured to transmit one or more signals 40 to the progressive load monitoring system 32 (e.g., resistance module 44) directly or via wearable device 12. The one or more signals 40 from the resistance equipment 26 may include data sensed, detected, or measured by the resistance equipment 26. Examples of the data may include the magnitude of the force, tension, resistance, or weight of the resistance equipment 26 and/or the number of repetitions (e.g., the number of the moving portion 28 being pushed, pulled, lifted, lowered, or rotated). In some examples, the resistance or weight of the resistance equipment 26 may be provided by the user as an input into the progressive load monitoring system 34.

In some examples, the progressive load monitoring system 34 may receive information indicative of a type of motion of a user to be monitored, for example, using the one or more wearable devices 12. The type of motion may include the use of the one or more exercise devices 20, 26 and/or the type, or level of the exercise devices 20, 26. When the exercise device is a resistance band 20, the progressive load monitoring system 34 may receive a resistance level of the resistance band 20. When the exercise device is a resistance equipment 26, the progressive load monitoring system 34 may receive a weight level of the resistance equipment 26.

Upon receiving, via the one or more sensors 16, 18 of the one or more wearable devices 12, information indicative of a speed and/or direction of a motion of the user, the progressive load monitoring system 34 may analyze the motion of the user based on the received information (e.g., information indicative of the speed and direction of the user’s motion and information indicative of the type of motion of the user to be monitored). For example, the analysis of the motion of the user may include calculating, by the force evaluator 46, a magnitude of a force applied to a body part of the user (e.g., a magnitude of a force that resists the motion) based on the speed of the motion, the direction of the motion, and/or information about the exercise device.

The analysis of the motion of the user may also include determining, by the force evaluator 46, speed values of the motion of the user in three different axes based on acceleration and/or orientation measured with sensors 16, 18. The analysis of the motion of the user may also include determining, by the force evaluator 46, a rotational velocity value for one or more planes based on the speed and direction of the motion.

In some examples, the force evaluator 46 may include one or more kinematic models 48, and the analysis of the motion may be performed using the one or more kinematic models 48. For example, the force evaluator 46 may determine the force applied by the exercise device on each portion of a user’s kinematic chain by using the kinematic models 48. The kinematic model 48 may represent links of a kinematic chain and include data representing the transfer of force between these links, data indicating relative motion of different links of the kinematic chain, and other types of data that facilitates mathematical representation of moving elements (e.g., joints, limbs, etc.).

In some examples, the progressive load monitoring system 34 may determine, based on the analysis, whether the motion of the user satisfies a predetermined motion target. The predetermined motion target may include at least one of a target speed, a range of target speeds, or a threshold number of repetitions.

In some examples, the progressive load monitoring system 34 may determine the target speed or target range of speeds based on the user’s motion and/or data from the motion module 42 and/or resistance module 44 (e.g., by identifying a motion or exercise being performed by the user by accessing a database storing previously-performed movements). The target speed or range of speeds may be different among three different axes, or planes of rotations. In some examples, the progressive load monitoring system 34 may determine the target speed or range of speeds based on the level of force, resistance, or weight required for a given exercise device.

Referring to FIG. 2, when the user exercises using the resistance band 20, the progressive load monitoring system 34 may deliver feedback to promote motion in the x-axis and limit motion in the y and z axes. For example, the progressive load monitoring system 34 may require that the speed of the motion of the user in the x-axis be equal to or greater than a first predetermined target speed. The progressive load monitoring system 34 may also require that the speed of a motion of the user in the y and z axes be equal to or less than a second predetermined target speed. The first predetermined target speed may be greater than the second predetermined target speed. For example, the first predetermined target speed may be at least five times or ten times greater than the second predetermined target speed.

Referring back to FIG. 3, when the user exercises using the resistance equipment 26, the progressive load monitoring system 34 may deliver feedback to promote motion in the z-axis and to limit motion in the x and y axes. For example, the progressive load monitoring system 34 may require that the speed of a motion of the user in the z-axis be equal to or greater than a third predetermined target speed. The progressive load monitoring system 34 may also require that the speed of a motion of the user in the x and y axes be equal to or less than a fourth predetermined target speed. The third predetermined target speed may be greater than the fourth predetermined target speed. For example, the third predetermined target speed may be at least five times or ten times greater than the fourth predetermined target speed.

In some examples, the progressive load monitoring system 34 may set a maximum speed for the motion of the user, for example, for each axis. In some examples, when the motion of the user is out of a target speed range (e.g., too fast, too slow, etc.), the progressive load monitoring system 34 may provide a real-time feedback to the user to correct the motion (e.g., decrease or increase the speed of the motion in a specific direction). For example, the progressive load monitoring system 34 may transmit one or more feedback signals to the wearable device 12, which displays the feedback from progressive load monitoring system 34 on the display 14.

In some examples, the progressive load monitoring system 34 may output a flag indicating a counted motion (e.g., correct motion) and/or a flag indicating a non-counted motion (e.g., incorrect motion). The progressive load monitoring system 34 may consider the counted motion for the threshold number of repetitions, and/or ignore the non-counted motion for the threshold number of repetitions. For example, when a user performs three non-counted motions and five counted motions, the progressive load monitoring system 34 may consider that there were only five repetitions (by ignoring the three non-counted motions) when determining whether the number of repetitions of the motions by the user is equal to or greater than the threshold number of repetitions.

In some examples, the feedback and recommendation module 50 may be configured to transmit one or more signals 52 to the user via the wearable device 12. The one or more signals 52 may include a recommendation to the user. The recommendation may include a suggestion or other identification for a motion (e.g., prescribed movements for the user wearing the wearable device 12).

In some examples, responsive to determining that the motion of the user satisfies the predetermined motion target, the progressive load monitoring system 34 may provide a recommendation to the user via the wearable device 12. The recommendation may include a suggestion for a motion that may require an increased magnitude of a force (higher level of resistance or weight for the resistance band 20 or resistance equipment 26). For example, the progressive load monitoring system 34 may recommend an increase or a decrease in the magnitude of force for a future motion based on the determination that the speeds of the motion of the user and/or the number of motion repetitions correspond to the target speed, the target range of speeds, and/or the threshold number of repetitions.

In some examples, the progressive load monitoring system 34 may correlate the force/tension of the movement being performed (assuming that the force value is generated by the exercise device) with a baseline of the same movement without the influence/use of the exercise device. In some examples, the progressive load monitoring system 34 may determine/calculate the applied force by the user for all or partial body parts (e.g., joints/limbs/muscles), while the respective body parts are under the influence of external force applied by the movement using the exercise device. This force value can be used as an indicator of the user’s strength level.

In some examples, based on the monitored and calculated data related to the user’s movements (e.g., the movement metrics, such as sets and repetitions of the movements, a range of motions, speed, acceleration, rotational velocity, etc.), the progressive load monitoring system 34 may determine a strength level of the user. For example, the system may analyze the user’s movements in three dimensions (e.g., along the x, y, z axes), and calculate or otherwise determine related parameters (e.g., acceleration, rotational velocity, etc.) in each axis or plane. In some examples, the progressive load monitoring system 34 may measure all of the user movement metrics for each strength level, and determine/calculate a pain score for each strength level. In some examples, the pain score may be proportional to the strength level.

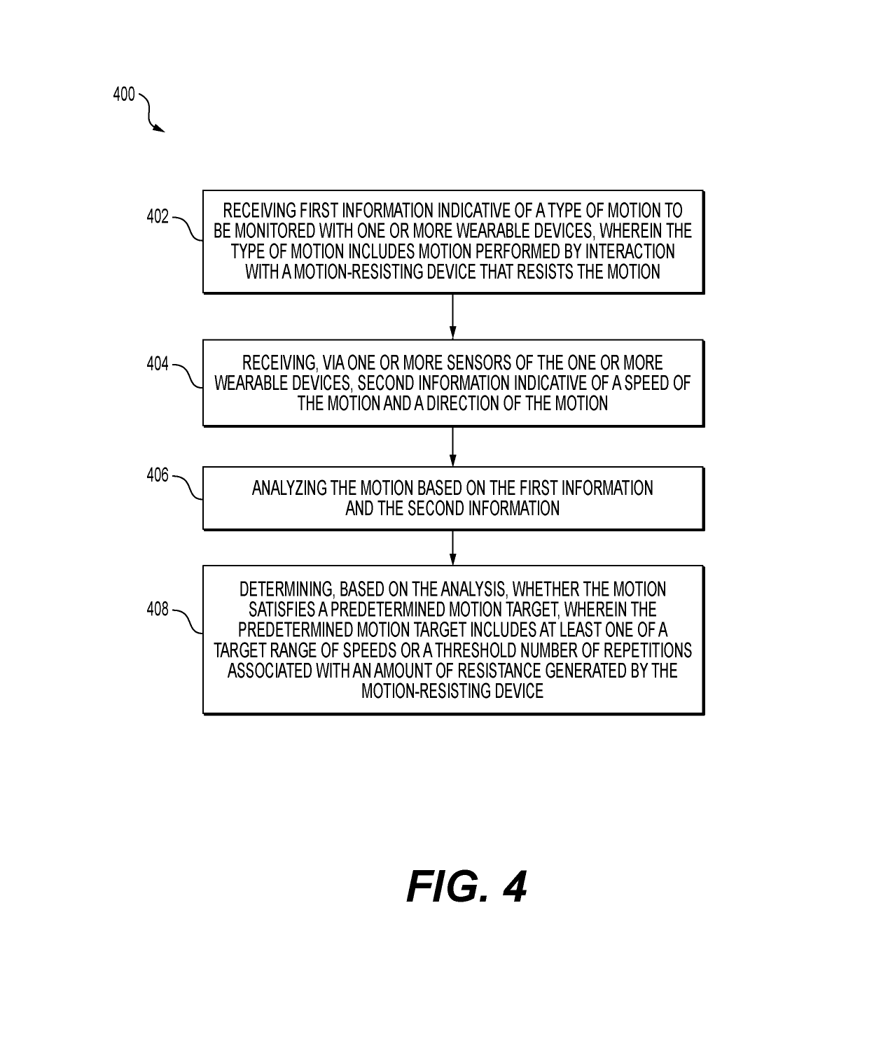

FIG. 4 illustrates a flow diagram of an example method 400 for progressive load monitoring according to some example embodiments of the present disclosure. Although the example method 400 is described with reference to the flow diagram illustrated in FIG. 4, it will be appreciated that many other methods of performing the acts associated with the method may be used. For example, the order of some of the blocks may be changed, certain blocks may be combined with other blocks, and some of the blocks described are optional. In some examples, the method 400 may be implemented using, for instance, a chip set or a device (e.g., progressive load monitoring system 32) including one or more processors and one or more memory devices.

In the illustrated example, the progressive load monitoring system 32 may receive first information indicative of a type of motion of a user to be monitored with one or more wearable devices 12, where the type of motion includes a use of an exercise device (block 402). The type of motion may include the use of the one or more exercise devices 20, 26 and/or the type or level of the exercise devices 20, 26. In some examples, the first information may further include information about the body parts of the user using the exercise device, and resistance/weight level of the exercise device.

In some examples, the progressive load monitoring system 32 may receive, via one or more sensors 16, 18 of the one or more wearable devices 12, second information indicative of a speed of a motion of a user and a direction of the motion (block 404). One or more wearable devices 12 may be used to monitor or detect the speed and/or direction of a motion of the user. In some examples, the one or more wearable devices 12 may be attached to the user’s wrist or arm. In other examples, the one or more wearable devices 12 may be attached to any other suitable body part of the user (e.g., leg, foot, chest, face, head, etc.).

In some examples, the progressive load monitoring system 32 may analyze the motion of the user based on the first information and the second information (block 406). For example, the progressive load monitoring system 32 may determine speed values of the motion of the user in three different axes based on the speed of the motion of the user. The progressive load monitoring system 32 may also determine a rotational velocity value for one or more planes based on the speed and direction of the motion.

In some examples, the progressive load monitoring system may determine, based on the analysis, whether the motion of the user satisfies a predetermined motion target, wherein the predetermined motion target includes at least one of a target range of speeds or a threshold number of repetitions (block 408). In some examples, the progressive load monitoring system 34 may determine the target speed or target range of speeds based on the user’s motion and/or data from the motion module 42 and/or resistance module 44. The target speed or range of speeds may be different among three different axes, or planes of rotations. In some examples, the progressive load monitoring system 34 may determine the target speed or range of speeds based on the level of force, resistance, or weight required for a given exercise device.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, or in a different order. The order of steps presented are only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the disclosure being indicated by the following claims.

What is claimed is:

1. A system for progressive load monitoring with wearable devices, the system comprising:
a memory storing instructions;
one or more processors that, when executing the instructions, perform operations including:
receiving first information indicative of a type of motion of a user to be monitored with one or more wearable devices, wherein the type of motion includes motion performed by interaction with a motion-resisting device that resists the motion;
receiving, via one or more sensors of the one or more wearable devices, second information indicative of a speed of the motion and a direction of the motion;
analyzing the motion based on the first information and the second information; and
determining, based on the analysis, whether the motion satisfies a predetermined motion target, wherein the predetermined motion target includes at least one of a target range of speeds or a threshold number of repetitions associated with an amount of resistance generated by the motion-resisting device.

2. The system of claim 1, wherein the motion-resisting device comprises a resistance band, and the first information further includes a resistance level of the resistance band.

3. The system of claim 1, wherein the motion-resisting device comprises a weight, and the first information further includes a level of the weight.

4. The system of claim 1, wherein the analysis of the motion of the user comprises calculating a magnitude of a force of tension applied to a body part of the user based on the speed of the motion, the direction of the motion, and information about the motion-resisting device.

5. The system of claim 1, wherein the analysis of the motion comprises determining speed values of the motion in three different axes based on the speed of the motion.

6. The system of claim 5, wherein the target range of speeds comprises a set of target speed ranges for each of the three different axes.

7. The system of claim 1, wherein the analysis of the motion comprises determining a rotational velocity value for one or more planes based on the speed and direction of the motion.

8. The system of claim 1, wherein the one or more sensors comprise at least one of an accelerometer or a gyroscope.

9. The system of claim 1, wherein the operations include, responsive to determining that the motion of the user satisfies the predetermined motion target, providing a display identifying a motion that requires an increased magnitude of force.

10. The system of claim 1, wherein the operations include outputting a flag indicating a counted motion or a flag indicating a non-counted motion.

11. The system of claim 10, wherein the operations include:
considering the counted motion for the threshold number of repetitions; and
ignoring the non-counted motion for the threshold number of repetitions.

12. A method for progressive load monitoring with wearable devices, the method comprising:
receiving first information indicative of a type of motion to be monitored with one or more wearable devices, wherein the type of motion includes motion performed by interaction with a motion-resisting device that resists the motion;
receiving, via one or more sensors of the one or more wearable devices, second information indicative of a speed of the motion and a direction of the motion;
analyzing the motion based on the first information and the second information; and
determining, based on the analysis, whether the motion satisfies a predetermined motion target, wherein the predetermined motion target includes at least one of a target range of speeds or a threshold number of repetitions associated with an amount of resistance generated by the motion-resisting device.

13. The method of claim 12, wherein the motion-resisting device comprises a resistance band, and the first information further includes a resistance level of the resistance band.

14. The method of claim 12, wherein the motion-resisting device comprises a weight, and the first information further includes a level of the weight.

15. The method of claim 12, wherein the analysis of the motion comprises calculating a magnitude of a force of tension applied to a body part of the user based on the speed of the motion, the direction of the motion, and information about the motion-resisting device.

16. The method of claim 12, wherein the analysis of the motion comprises determining speed values of the motion in three different axes based on the speed of the motion.

17. The method of claim 16, wherein the target range of speeds comprises a set of target speed ranges for each of the three different axes.

18. The method of claim 12, wherein the analysis of the motion comprises determining a rotational velocity value for one or more planes based on the speed and direction of the motion.

19. The method of claim 12, further comprising, responsive to determining that the motion satisfies the predetermined motion target, providing a display identifying a motion that requires an increased magnitude of force.

20. A non-transitory computer readable storage medium, having instructions stored therein, which when executed by one or more processors, cause the one or more processors to:
receive first information indicative of a type of motion of a user to be monitored with one or more wearable devices, wherein the type of motion includes motion performed by interaction with a motion-resisting device that resists the motion;
receive, via one or more sensors of the one or more wearable devices, second information indicative of a speed of the motion and a direction of the motion;
analyze the motion based on the first information and the second information; and
determine, based on the analysis, whether the motion satisfies a predetermined motion target, wherein the predetermined motion target includes at least one of a target range of speeds or a threshold number of repetitions associated with an amount of resistance generated by the motion-resisting device.
